(19) United States
(12) Patent Application Publication
(10) Pub. No.: US 2013/0336445 A1
(43) Pub. Date: Dec. 19, 2013

(54) ROI SELECTION FOR IMAGING APPARATUS

(71) Applicants: William J. Sehnert, Fairport, NY (US); Xiaohui Wang, Pittsford, NY (US); Samuel Richard, Rochester, NY (US)

(72) Inventors: William J. Sehnert, Fairport, NY (US); Xiaohui Wang, Pittsford, NY (US); Samuel Richard, Rochester, NY (US)

(73) Assignee: Carestream Health, Inc.

(21) Appl. No.: 13/608,163

(22) Filed: Sep. 10, 2012

Related U.S. Application Data
(63) Continuation-in-part of application No. 13/523,264, filed on Jun. 14, 2012.

(51) Int. Cl. A61B 6/02; G01N 23/04

(52) U.S. Cl. 378/42; 378/62

(57) ABSTRACT

A method for acquiring a sequence of fluoroscopic images of a subject acquires and displays a basis image from a fluoroscopic imaging system. A region of interest is defined within the displayed basis image in response to one or more viewer instructions entered on the displayed basis image. One or more signals are generated that adjust the position of one or more components of the fluoroscopic imaging system according to the one or more viewer instructions.

### CROSS REFERENCE TO RELATED APPLICATIONS

This is a Continuation-In-Part of U.S. Ser. No. 13/523,264 filed Jun. 14, 2012 in the names of Sehnert et al. entitled “REGION-SELECTIVE FLUOROSCOPIC IMAGE COMPRESSION”.

### FIELD OF THE INVENTION

The invention relates generally to the field of medical imaging; more particularly to a method for control of components of a fluoroscopic imaging apparatus according to identification of a region of interest.

### BACKGROUND OF THE INVENTION

Fluoroscopy provides near real-time visualization of internal anatomy of a patient, with the ability to monitor dynamic processes, including tracking the relative motion of various types of features such as probes or other devices, fluids, and structures. Fluoroscopy is used, for example to help in diagnosis and to position the patient for subsequent image recording or to position and manipulate various types of devices for interventional procedures.

The block diagram of FIG. 1 shows components in the imaging path of a conventional fluoroscopy system 10 for obtaining images of a patient 14 or other subject. Radiation from an x-ray source 20 that typically uses a collimator 22 and filtration 24 is directed through a patient 14 to an image intensifier 30. Generally a grid 32 is provided. A camera 40 then captures successive video frames from the x-ray exposure and generates images that are displayed on a display monitor 44.

To reduce the exposure of the patient to ionizing radiation, conventional fluoroscopy practices use the collimator 22 to limit the size of the exposure field as much as possible. Adjustments to collimator 22 are made using an initial “scout image” to ascertain how well the radiation beam is centered and how much adjustment of the collimators can be allowed in order to direct radiation to the region of interest (ROI) for a particular patient 14. The practitioner views the scout image and makes adjustments accordingly, then begins the active imaging sequence for fluoroscopy. This procedure is time-consuming and approximate, sometimes requiring repetition of the adjustment to correct for error. Moreover, movement of the patient or ongoing progress of a contrast agent or probe or other device can cause the ROI to shift, requiring that the imaging session be repeatedly paused in order to allow for collimator readjustment.

As digital radiography (DR) imaging receivers steadily improve in image quality and acquisition speed, it is anticipated that these devices can be increasingly employed not only for conventional radiography imaging, but also for fluoroscopy applications, effectively eliminating the need for the dedicated image intensifier hardware used with conventional fluoroscopy systems such as that shown in FIG. 1. The problems of collimator adjustment, however, including some amount of guesswork and inaccuracy, remain.

Thus, it can be seen that there is a need for methods that enable accurate and facile collimator adjustment when using DR receivers for imaging in fluoroscopy systems.

### SUMMARY OF THE INVENTION

An object of the present invention is to address the need for more efficient and accurate ways to obtain suitable collimator settings for fluoroscopy applications. The methods and apparatus provided utilize the capability of the DR fluoroscopy system to display results and to obtain operator instructions directly from the display, thereby allowing the ROI to be readily adjusted by a practitioner during an x-ray scan sequence.

According to an embodiment of the present invention, there is provided a method for acquiring a sequence of fluoroscopic images of a subject, the method comprising: acquiring and displaying a basis image from a fluoroscopic imaging system; defining a region of interest within the displayed basis image in response to one or more viewer instructions entered on the displayed basis image; and generating one or more signals that adjust the position of one or more components of the fluoroscopic imaging system according to the one or more viewer instructions.

These objects are given only by way of illustrative example, and such objects may be exemplary of one or more embodiments of the invention. Other desirable objectives and advantages inherently achieved by the disclosed invention may occur or become apparent to those skilled in the art. The invention is defined by the appended claims.

### BRIEF DESCRIPTION OF THE DRAWINGS

The foregoing and other objects, features, and advantages of the invention will be apparent from the following more particular description of the embodiments of the invention, as illustrated in the accompanying drawings. The elements of the drawings are not necessarily to scale relative to each other.

FIG. 1 is a schematic block diagram showing components of a conventional fluoroscopic imaging apparatus.

FIG. 2A is a schematic block diagram showing components of a fluoroscopic imaging apparatus using wired image data transmission.

FIG. 2B is a schematic block diagram showing components of a fluoroscopic imaging apparatus using wireless image data transmission.

FIG. 3 is a schematic block diagram that shows functional components of a fluoroscopy capture and display apparatus according to embodiments of the present invention.

FIG. 4A is a plan view that shows a fluoroscopy image of a patient's head.

FIG. 4B is a view of the image of FIG. 4A showing a rectangular region of interest, defined according to an embodiment of the present invention.

FIG. 5 is a diagram that shows successive image frames in a fluoroscopy imaging sequence.

FIG. 6A is a view of an operator interface for defining the region of interest for a fluoroscopy imaging sequence using a rectangle.

FIG. 6B is a view of an operator interface for defining the region of interest for a fluoroscopy imaging sequence using a mask.

FIG. 6C is a view of an operator interface for defining the region of interest for a fluoroscopy imaging sequence using a device or object.

FIG. 6D is a view of an operator interface for defining the region of interest for a fluoroscopy imaging sequence using a collimator setting.

FIG. 7 is a logic flow diagram that shows steps for applying a selective compression sequence according to an embodiment of the present invention.

FIG. 8A is a view of a display screen showing a shift in position of the region of interest according to movement of an object or device.

FIG. 8B is a view of a display screen showing a shift in position of the region of interest according to a change in operator focus of attention.

FIG. 9A is a logic flow diagram that shows steps for transmitting image data from region of interest and background portions of an image at different rates.

FIG. 9B shows timing diagrams for transmitting image data from region of interest and background portions of an image at different rates, as described with reference to FIG. 9A.

FIG. 10 is a schematic diagram that shows components for control of collimator sizing and positioning according to an embodiment of the present invention.

FIG. 11 is a schematic diagram that shows components for control of source and detector positioning according to an embodiment of the present invention.

FIG. 12 is a logic flow diagram that shows a sequence of steps for controlling devices of the fluoroscopy system.

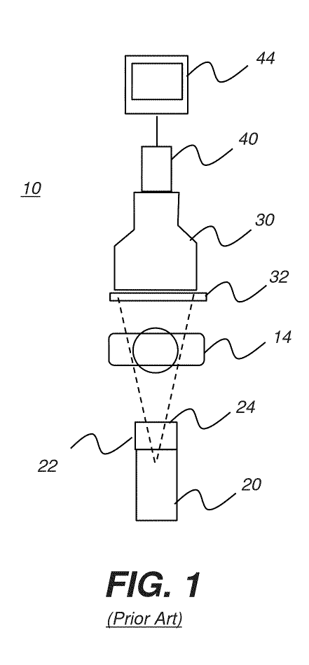

### DETAILED DESCRIPTION OF THE INVENTION

The following is a detailed description of the preferred embodiments of the invention, reference being made to the drawings in which the same reference numerals identify the same elements of structure in each of the several figures.

Where they are used, the terms “first”, “second”, and so on, do not necessarily denote any ordinal, sequential, or priority relation, but are simply used to more clearly distinguish one element or set of elements from another, unless specified otherwise. The term “pixel” has its standard meaning, referring to a picture element, expressed as a unit of image data.

In the context of the present disclosure, the terms “viewer”, “operator”, and “user” are considered to be equivalent and refer to the viewing practitioner or other person who views and manipulates an x-ray image, such as a fluoroscopic image, on a display monitor. A “viewer instruction” can be obtained from explicit commands entered by the viewer on the surface of the display or may be implicitly obtained or derived based on some other user action, such as setting up or initiating an exposure or making a collimator adjustment, for example.

In the context of the present invention, the terms “near video rate” and “near real-time” relate to the response time for image data display. For fluoroscopy, because of detector response limitations and because it is beneficial to help reduce radiation levels, what is considered real-time or near-real-time video presentation is generally at a slower frame refresh rate than rates used for conventional video imaging. Thus, in the context of fluoroscopy imaging for example, a useful “near real-time” refresh rate is at least about 1 or more frames per second.

The term “highlighting” for a displayed feature has its conventional meaning as is understood to those skilled in the information and image display arts. In general, highlighting uses some form of localized display enhancement to attract the attention of the viewer. Highlighting a portion of an image, such as an individual organ, bone, or structure, or a path from one chamber to the next, for example, can be achieved in any of a number of ways, including, but not limited to, annotating, displaying a nearby or overlaying symbol, outlining or tracing, display in a different color or at a markedly different intensity or gray scale value than other image or information content, blinking or animation of a portion of a display, or display at higher resolution, sharpness, or contrast.

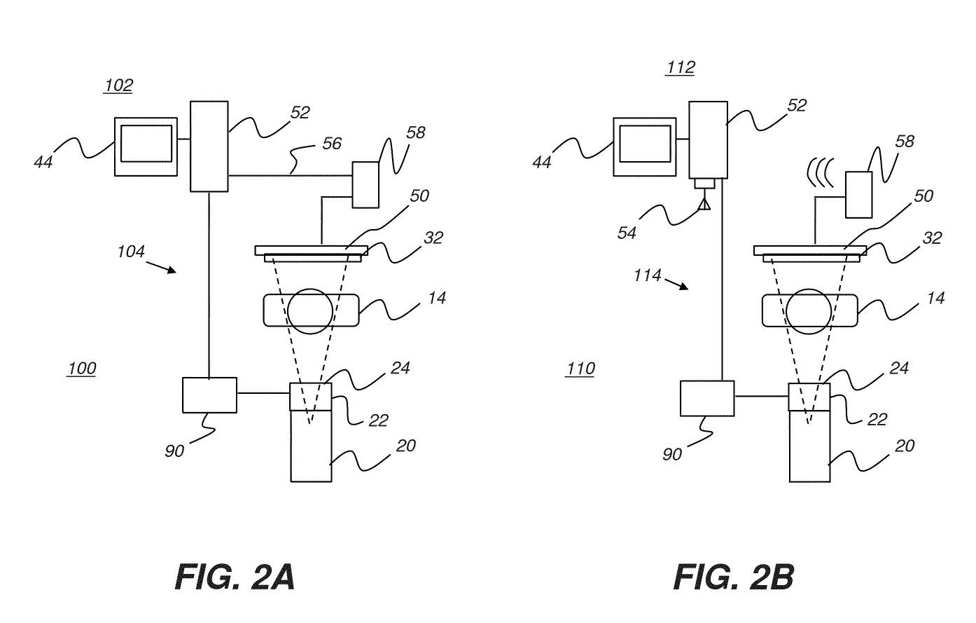

Embodiments of the present invention enable the use of a digital radiography (DR) receiver as the digital image receiver for receiving radiation in the fluoroscopy system and for generating, processing, and transmitting the received image data, as image pixels (picture elements), to a display apparatus for fluoroscopic display. FIGS. 2A and 2B respectively show two general arrangements of system components for a fluoroscopy system 100 that uses an interconnect cable 56 for image data transmission and a fluoroscopy system 110 that employs wireless transmission of image data.

FIG. 2A illustrates fluoroscopy system 100 having a fluoroscopy capture apparatus 104 that includes DR receiver 50 and an image processing unit 58 that obtains and processes the image data from detector 50 and transmits the processed image data to a host processor 52 through an interconnect cable 56 for providing the image data to a fluoroscopy display apparatus 102 that includes a display monitor 44. Host processor 52 is a computer or workstation or other logic and control processor that obtains the processed fluoroscopy image data and displays the obtained images at near-video rates to the practitioner or other viewer. An imaging controller 90 generates signals that control various aspects of operation of fluoroscopy capture apparatus 104, including the dimensions and placement of the collimator 22 opening, as described in more detail subsequently.

FIG. 2B shows fluoroscopy system 110 that has a fluoroscopy capture apparatus 114 in which image processing unit 58 provides the processed image data of a subject to a fluoroscopy display apparatus 112 in wireless form. Host processor 52 has a wireless receiver element 54 for providing the image data to fluoroscopy display apparatus 112 for viewing on display monitor 44.

For both FIG. 2A and FIG. 2B embodiments, image processing unit 58 may be integrated into DR receiver 50 or may be a separate processor apparatus. Image processing unit 58 may be a dedicated microprocessor, host processor, or other type of computer device, including a device that performs logic instructions that have been encoded in hardware.

An aspect in obtaining processed image data of the subject at near video rates relates to the need for both high-speed data access between DR receiver 50 and image processing unit 58 and high data transmission rates from image processing unit 58 to host processor 52 (FIGS. 2A, 2B). It is noted that this aspect is more pronounced with the wireless transmission of fluoroscopy system 110 in FIG. 2B, since wireless rates are generally slower than data rates with a hard-wired connection and since wireless transmission can be further hindered by intermittent noise and interference. Thus, methods for compacting the image data as much as possible offer one way to help alleviate the potential data transmission bottleneck that can occur with either wired or wireless transmission.

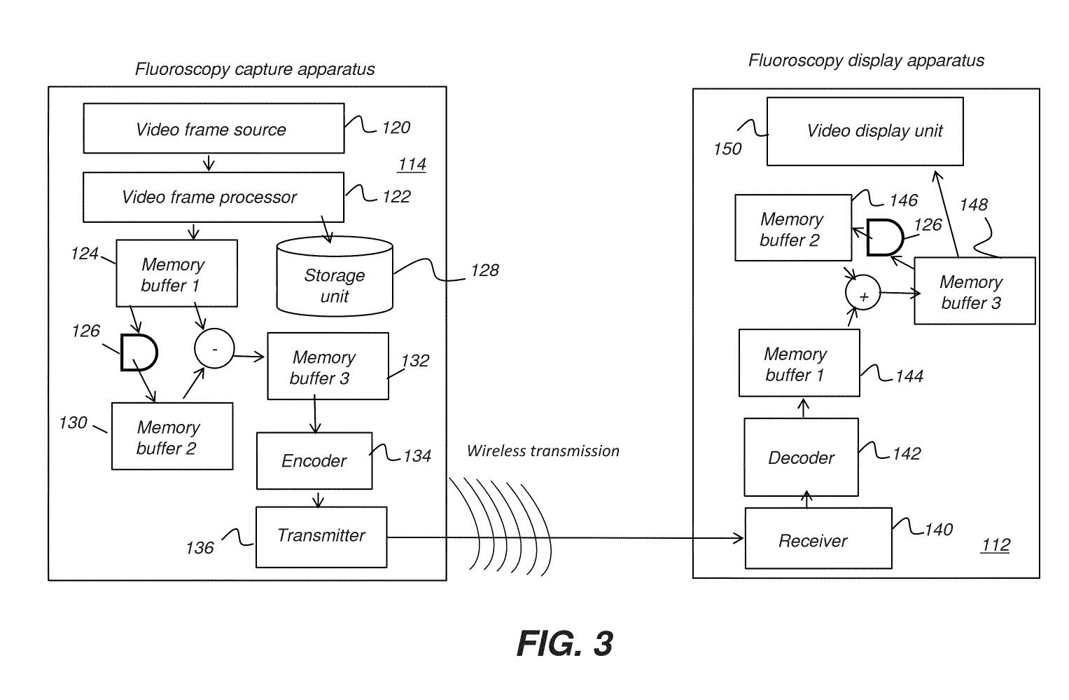

One method for reducing the bulk amount of data that must be transferred determines the differences between two successive frames and provides only the data that is indicative of the difference. The block diagram of FIG. 3 gives a functional overview of components for wireless transmission in the embodiment of fluoroscopy system 110 shown in FIG. 2B that uses difference information between successive image frames. At fluoroscopy capture apparatus 114, a video frame source 120 includes the DR receiver 50 components that obtain the digital data that is representative of the received radiation transmitted through patient 14 or other subject. A video frame processor 122, provided in image processing unit 58 in the FIGS. 2A and 2B embodiments, processes the received frame of image data for rendering quality and outputs the processed frame into a memory buffer 124. Utilities that can be used for improving rendering quality include, for example, tone scale adjustment, unsharp masking, and other functions. Optionally, the image data is also sent to a storage unit 128 for longer term archival. A first memory buffer 124 contains the current image frame. A second memory buffer 130 contains image content for the preceding frame. Processing compares memory buffers 124 and 130 to generate difference data between successive image frames and store this in a third memory buffer 132. The image data contents of third memory buffer 132 are then provided to an encoder 134 for compression and to a transmitter 136 for data transmission. This provides compressed fluoroscopy data for transmission to fluoroscopy display apparatus 112. For processing the next frame of image data, after a delay 126, data from memory buffer 124 becomes memory buffer 130 data.

Continuing with the sequence shown in FIG. 3, the transmitted data goes to fluoroscopy display apparatus 112. A receiver 140 receives the compressed image data and provides this data to a decoder 142. The decoded data then goes to a memory buffer 144 as a difference image. This image data is combined with image data for the previous frame that is in a memory buffer 146 to form image data that is then stored in a memory buffer 148. Image data from memory buffer 148 is then provided to a video display unit 150 for display on the display monitor and to memory buffer 146 for processing the next frame. Delay 126 is provided between transfer of data from memory buffer 148 to memory buffer 146.

With respect to the sequence described with reference to FIG. 3, it should be noted that the first image frame is handled differently, stored in the appropriate memory buffer to provide initial reference data for subsequent processing. Although described primarily with reference to the wireless embodiment of FIG. 2B, the same basic processing sequence used within capture apparatus 114 and display apparatus 112 in FIG. 3 can also be used in the hard-wired embodiment of FIG. 2A.

The difference scheme used in the sequence described with reference to FIG. 3 helps to reduce the amount of image data that is transferred in wired or wireless form. Difference data can be transmitted for either or both the region of interest or the background region. However, there can still be a considerable amount of data to be transferred. Moreover, not all of the transferred data may be as important/relevant for the clinical or diagnostic function. There may be some image data for which compression is not desirable, where compression results in any loss of image content. Some types of image compression are lossy, so that some amount of image data can be compromised when compression is used. The resulting loss of data may make compression undesirable for some portion of the image content. At least one embodiment of the present method addresses this by allowing the viewer to define regions of interest that are of particular relevance, where loss of image content may be detrimental/undesirable to the function for which fluoroscopy is being used. Image data content that lies outside this region of interest may then be subjected to some amount of lossy compression without sacrificing clinical or diagnostic value. Image data within the region of interest is then transmitted without compression, or using compression methods that are lossless.

Image data compression techniques can be lossless or lossy and embodiments of the present invention can employ both types of compression for different types of image content. Lossless image data compression techniques include methods such as Run-Length Encoding (RLE) that eliminates some amount of data redundancy within a stream or sequence of data code values. Other, more sophisticated types of lossless compression for image data known to those skilled in the image processing arts include entropy coding, dictionary encoding techniques, and LZW (Lempel-Ziv-Welch) compression. File formats including JPEG (Joint Photographic Experts Group) LS, TIFF (Tagged Image File Format), GIF (Graphics Interchange Format), PNG (Portable Network Graphics), and other standard types of file formats often provide or support some measure of lossless compression encoding, with techniques and options for lossless encoding of the corresponding image data.

One general group of lossy encoding strategies known to those skilled in the image representation and storage arts uses transform coding or transform-based methods; JPEG and JPEG2000 are in this category. Another general type of encoding is bit field encoding, such as that used in BMP (BitMaP file format) encoding. Predictive encoding is yet another general type of encoding, including JPEG lossless and JPEG-LS encoding. No compression, that is, sending the data uncompressed, is also considered to provide a lossless encoding in the context of the present disclosure.

Lossy image data compression techniques can considerably reduce the amount of data for a given image but allow some loss of information, such as image content that is relatively less perceptible to the human eye. Standard image compression used with JPEG format is lossy and compresses image data by approximation techniques such as by rounding image data values where visual information is less important. Wavelet compression is another lossy compression type that can yield satisfactory results for medical images. Any type of lossy data compression or data format that compromises any of the image data is considered to provide a lossy encoding.

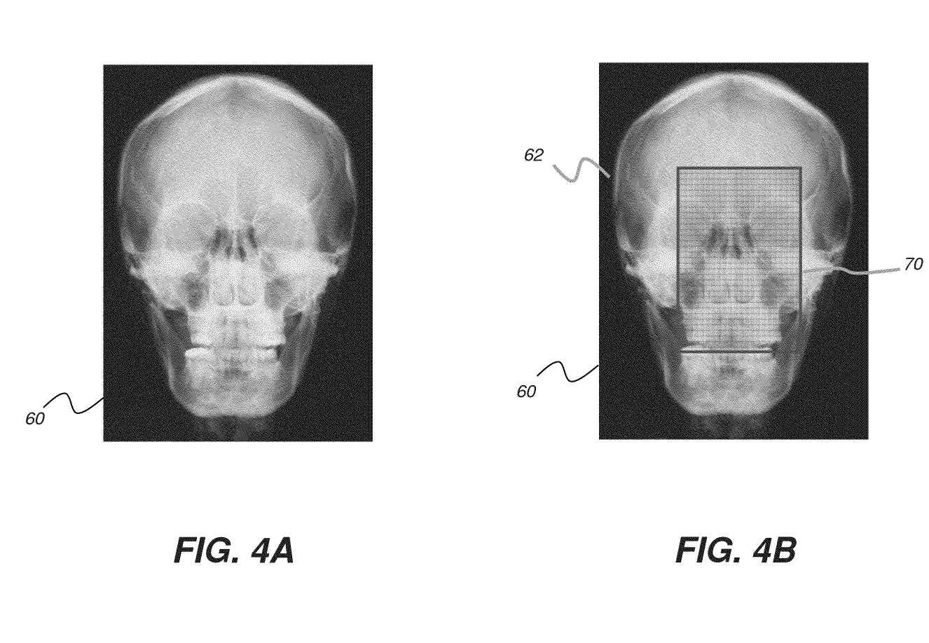

FIG. 4A shows a fluoroscopy image 60 that includes a patient's head. For a particular procedure, only a portion of the patient's head is of interest. As shown in FIG. 4B, there is a region of interest (ROI) 70, identified as a rectangular area in this example. The balance of image 60, exclusive of region of interest 70, is a background region 62.

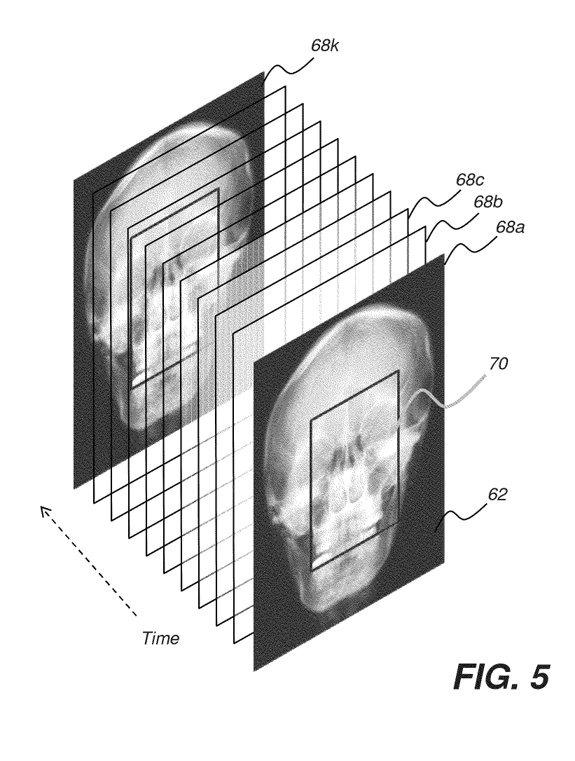

FIG. 5 shows a series of successive image frames 68a, 68b, 68c . . . 68k in a small portion of an example fluoroscopy sequence. As can be seen, the same anatomy is imaged in each image frame. Of primary interest to the practitioner is region of interest 70 within each frame; background region 62 is of less value for the procedure that is being performed. For this reason, embodiments of the present method allow different types of image processing and image data compression and transmission for the two (or more) portions of the image, e.g., for region of interest 70 and background region 62. This allows the display of region of interest 70 at higher resolution and contrast than the display of background region 62, for example.

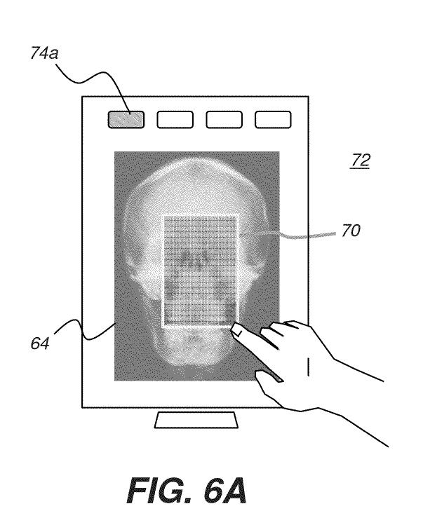

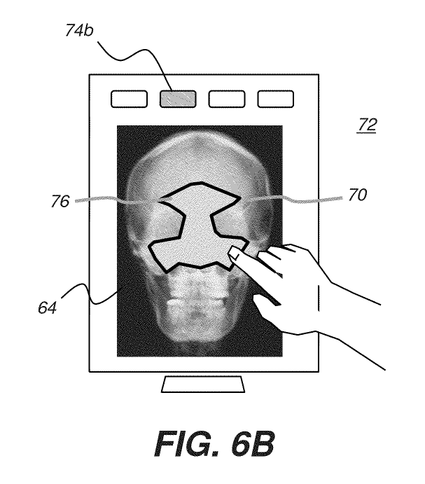

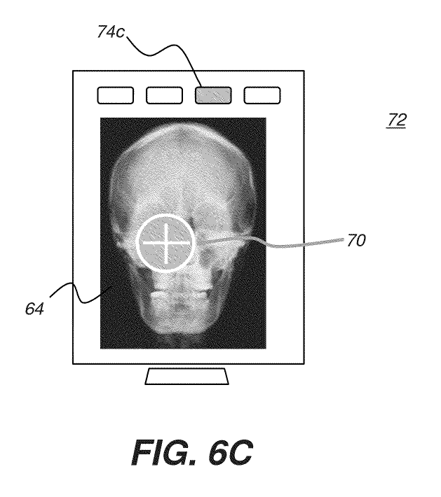

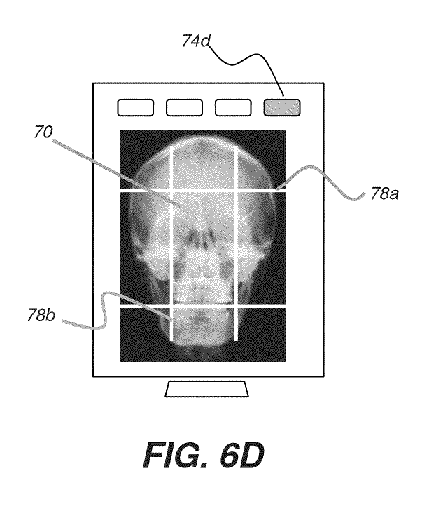

Regardless of the method that is employed for image compression and transmission, region of interest 70 is identified, relative to the image area of the digital detector or receiver, DR receiver 50 (FIGS. 2A, 2B). This can be done in a number of ways, such as those shown in the examples of FIGS. 6A through 6D.

Some type of viewer instruction or action is used to define the region of interest. FIGS. 6A and 6B show identifying region of interest 70 according to a viewer instruction entered/indicated on the operator interface, termed a Graphical User Interface (GUI) 72 on display monitor 44 (FIGS. 2A, 2B). In the example shown in FIG. 6A, a touch screen interface allows the viewer to outline region of interest 70 directly on a displayed basis image 64. Basis image 64 is a single fluoroscopy image that is optionally obtained as a part of initial setup for the fluoroscopy session. An optional control button 74a allows for entry of an operator instruction that enables rectangular outlining, or outlining using a circle or other appropriate geometric shape, onto the displayed basis image. In the example shown, the operator uses conventional interface actions to identify diagonal corners of a rectangle that defines region of interest 70 on basis image 64.

Given viewer entered instructions that identify the ROI, the imaging system then correlates the defined ROI with the corresponding image area of the digital radiography receiver. The use of a basis image is optional; various methods could be used to isolate ROI 70 from the balance of image 60 and to provide a mapping that relates one or more areas of the digital receiver to the ROI.

FIG. 6B shows definition of region of interest 70 using a mask 76 that is identified or defined by the user with reference to the basis image. Mask 76 may be selected from a series of standard masks, or may be edited or drawn free-form using a touch screen or other type of screen pointer that indicates points, basic shapes, or areas of the image. An operator instruction at a control button 74b specifies this function.

User tracing or placement of a shape that defines a region of interest relative to a basis image can be performed in a number of ways, using standard user interface tools and utilities, that include a touch screen or use of a computer mouse or stylus or other pointer. According to an alternate embodiment of the present method, an explicit user instruction that is entered with respect to a basis image is not needed for ROI identification. Instead, a default region of interest 70 is automatically assigned within the image, such as that portion of the image area centered in the middle of the display screen, for example. Utilities are then provided for performing functions such as panning or positional adjustment, sizing and scaling and other functions that may further re-define the region of interest according to viewer instruction.

The viewer instruction can thus identify specific points that define the region of interest or can instruct the system to utilize a default image area or a selected one of a set of default image areas for defining the region of interest.

The example of FIG. 6C shows another default arrangement that can be used. A viewer instruction entered on a control button 74c instructs the system to track a device or object, such as an instrument, camera, probe, needle, tube, or other object that is placed on or inserted into the patient anatomy being imaged. Region of interest 70 is defined in the vicinity of the tracked device or object and can have a default size, such as a given diameter about the object or device, or a viewer-defined size. For tracking an object, an initial calibration or setup procedure may be required for identifying the object and defining the size of the corresponding region of interest within which the object is centered.

The example illustrated in FIG. 6D shows another alternate embodiment in which the operator instruction, entered using a control button 74d, allows the system to define the boundaries of region of interest 70 according to the settings of collimator 22 blades (FIGS. 2A, 2B), as adjusted by the viewer. Collimator 22 typically provides either a circular region of variable diameter or a rectangular area of variable dimensions. In the example of FIG. 6D, a rectangular embodiment is shown. Lines 78a and 78b show the collimator blade settings, effectively providing a rectangular area as region of interest 70. On some systems, collimator blades are motor controlled, allowing the viewer to adjust and view settings for the area of interest as part of the overall equipment setup.

According to an alternate embodiment of the present method/apparatus, the operator can adjust collimator blade positions and observe blade repositioning directly on the display screen, allowing the system to adopt and change ROI boundaries according to blade settings. To obtain suitable coordinates for ROI identification, the imaging system detects the positions of collimator blades, and translates this positional information into corresponding coordinates on the detector for ROI identification.

Thus, in various ways, an ROI is identified, wherein the ROI maps to, or relates to, the image area of the digital detector of the imaging system. The viewer instruction that identifies/defines the ROI may be explicitly entered using the basis image as previously described, or may be inferred from a collimator or other adjustment. Alternately, the viewer instruction may simply be a command or instruction to prepare for obtaining images, thus prompting the imaging system to use a default ROI definition based on the type of image being obtained or based on sensed settings of the collimator, for example.

Once region of interest 70 is defined on the basis image, the viewer can enter an explicit instruction that indicates completion of this process. Alternately, the given settings are used automatically and exposure can begin. The specified region of interest settings are maintained until specifically adjusted by the viewer.

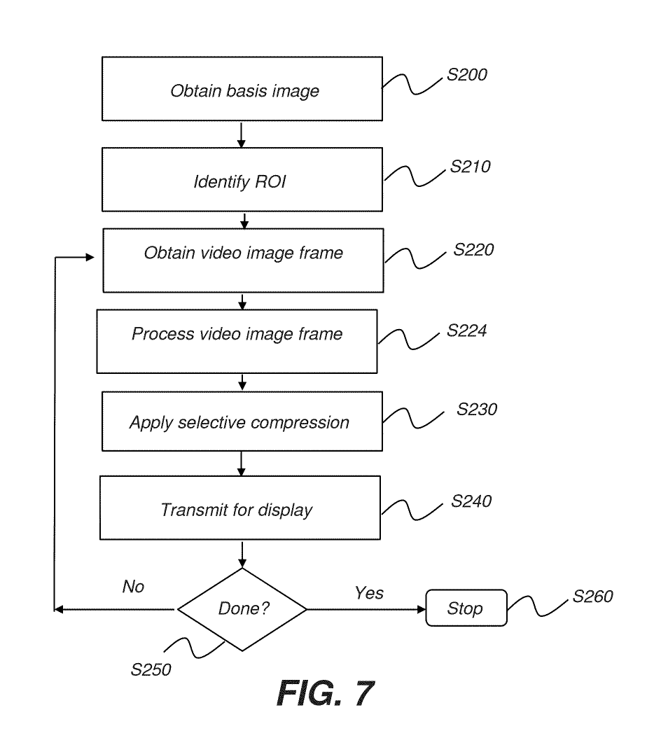

The logic flow diagram of FIG. 7 shows steps for fluoroscopic imaging according to an embodiment of the present invention. An optional obtain image step S200 obtains the basis image that is used for region of interest identification in some embodiments of the present invention. In an identify ROI step S210, the region of interest is identified, such as using procedures described with respect to FIGS. 6A through 6D. As noted previously, the ROI may be defined by default, without explicit operator markup on a basis image. The ROI may be automatically defined by default upon entry of an operator instruction to acquire a particular image.

Continuing with the FIG. 7 sequence, imaging proceeds with obtain video frame step S220, in which a frame of image data is acquired. The acquired image data is then processed in a processing step S224. Following image data processing, a selective compression step S230 then applies a first compression or a lossy compression to at least some of the background region pixels. A second compression or a lossless compression (including no compression, where this feature is used) is similarly applied to at least some region of interest pixels. A transmission step S240 transmits the encoded, processed image data to fluoroscopy display apparatus 102,112 (FIGS. 2A, 2B). A termination test S250 either proceeds if another frame is needed or moves to a termination step S260 to end the fluoroscopic imaging session.

Using the sequence described with reference to FIG. 7, the fluoroscopy system can selectively compress image data that is of less interest to the viewer, while providing no compression to data within the region of interest. According to an alternate embodiment of the present invention, two different compression levels are used. An aggressive, lossy compression is used for background region 62 content. A slightly lossy compression algorithm, allowing relatively less loss of image content by comparison with that applied for background region 62, is then used for region of interest 70. An algorithm is considered to be more or less lossy than another algorithm based on a measure of how much of the original processed image data is lost or modified when the compressed data is decompressed. Alternatively, the viewer can select (e.g., adjust) among data compression levels (e.g., image quality) for each of the region of interest and the region of less interest, respectively.

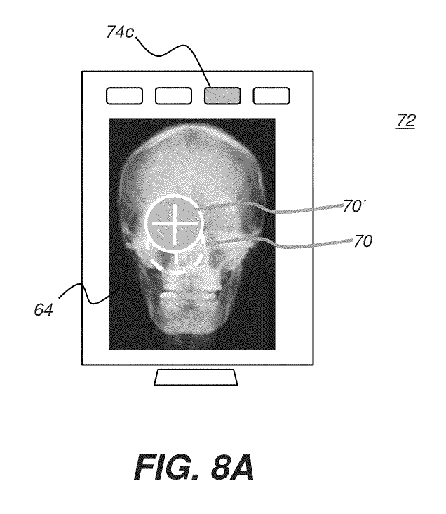

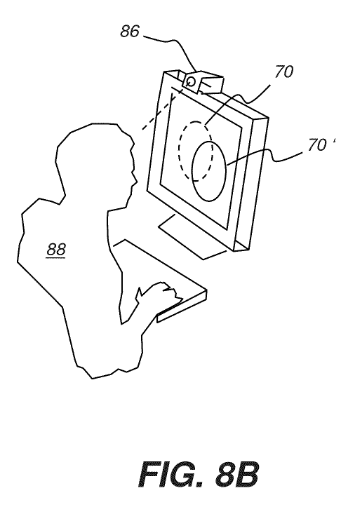

According to an embodiment of the present method, region of interest 70 can be shifted in position after it has been initially defined, during the fluoroscopy session. Referring to the example of FIG. 8A, a probe (not visible in the figure) is tracked and region of interest 70 is centered on the end of the probe, as indicated by crosshairs in FIG. 8A. Changing of probe position is tracked. As the probe is moved (upward in the example of FIG. 8A), a shifted region of interest 70′ is defined accordingly.

According to an alternate embodiment, as shown in FIG. 8B, region of interest 70 can be shifted according to a gesture or other indication from a viewer 88. In one arrangement, a gaze tracking mechanism is provided, observing viewer 88 attention using a camera 86 and signaling changes in viewing focus. As viewer 88 attention moves toward a different part of the image, region of interest 70 shifts to provide a shifted region of interest 70′.

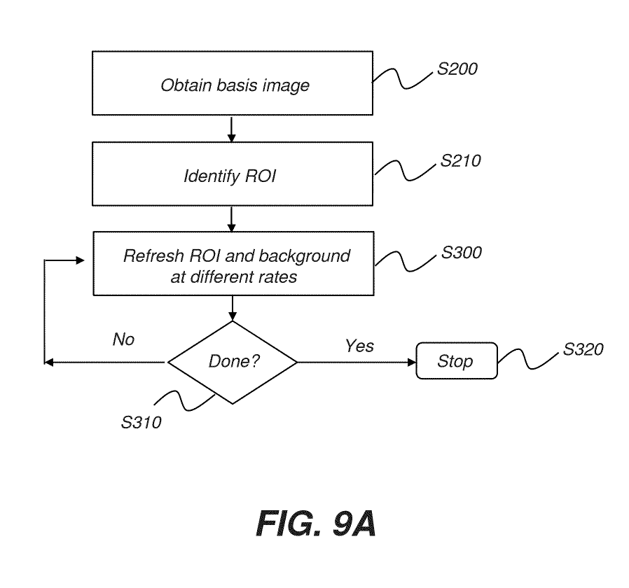

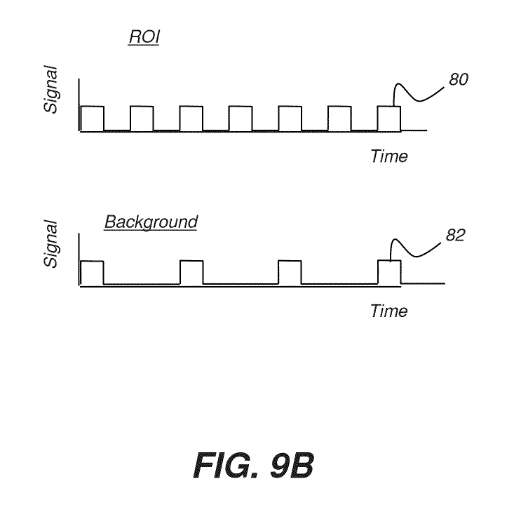

A different type of image data compression can be provided by effectively adjusting the timing of image update for the region of interest 70 so that its data refresh is more frequent than the update for background region 62. The logic flow diagram of FIG. 9A shows a sequence of steps for fluoroscopy imaging using this alternate technique. Optional obtain image step S200 and Identify ROI step S210 are the same as described earlier with reference to FIG. 7, allowing the viewer to define the region of interest that requires better resolution than background content, or assigning the region of interest by default, as previously described. A refresh step S300 provides a transmission sequence that refreshes the region of interest at a higher (faster) rate than it refreshes background content. FIG. 9B shows timing diagrams 80 and 82 that compare the refresh rates for region of interest 70 content and background region 62 content, respectively. By refreshing region of interest 70 content more often, the overall volume of image data that must be transmitted is significantly reduced, without significant impact on the quality of the displayed fluoroscopic image. Continuing with the sequence of FIG. 9A, a termination test S310 then either proceeds if another frame is needed or moves to a termination step S320 to end the fluoroscopic imaging session.

It is noted that once the region of interest is identified, the corresponding data content is handled appropriately for fluoroscopy display apparatus 102 (FIG. 2A) or 112 (FIG. 2B) at host processor 52. For a line of pixels, for example, one or more portions of the pixels may be part of the region of interest; other pixels in a line of pixels may be part of the background content. Pixel mapping to handle the different compression types can be relatively straightforward for the rectangular ROI 70 of FIG. 6A. For mask 76 of FIG. 6B, a binary mask is generated and provided to host processor 52, allowing the pixel data that is mapped to ROI and background content to be readily identified and appropriately handled for display.

According to an embodiment, different tone scales can be applied to the ROI and background content. This type of conditioning helps to visually differentiate background from ROI content for the viewer. Other types of perceptible image treatment can be provided over the full background or ROI areas, including use of different contrast or brightness levels, filtering, or use of color, for example.

According to an alternate embodiment, multiple levels of compression are used, depending on factors such as proximity to the region of interest. Displayed background content nearest the region of interest undergoes only slight compression, while content furthest from the region of interest is highly compressed.

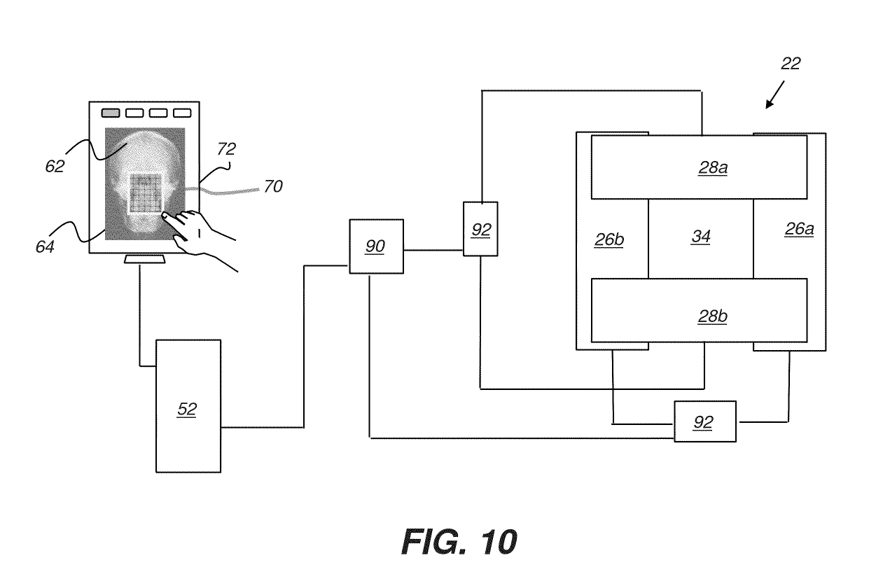

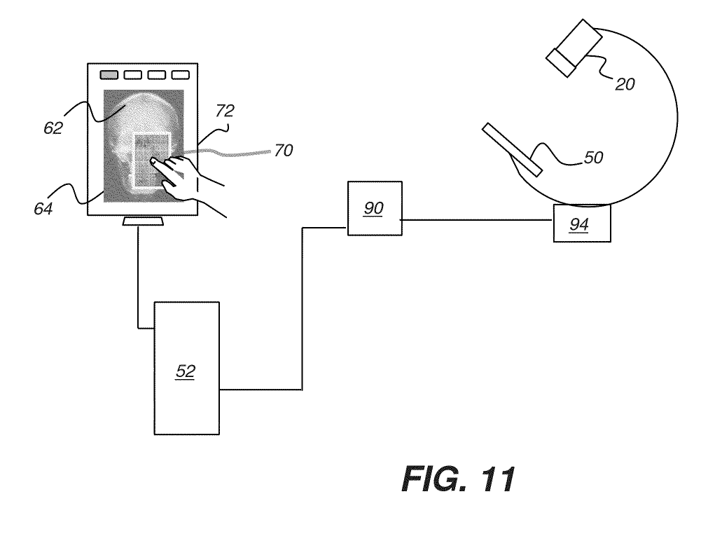

Described embodiments address the features to adjust and control aspects of operation of the fluoroscopy system according to the relative position of the region of interest (ROI), responding to operator instructions that are entered on the displayed image itself. The schematic diagram of FIG. 10 shows components for control of collimator sizing and positioning for a rectangular aperture according to an embodiment of the present invention. The operator defines ROI 70 as described previously, using a touch screen interface or other pointer on GUI 72 that positions an electronic cursor or other pointing element on the display. In response to a collimator sizing instruction, host processor 52 provides signals to imaging controller 90. Imaging controller 90, a logic processing and control component, such as a microprocessor or dedicated processor circuit, then energizes one or more actuators 92 to adjust the size and position of the collimator 22 aperture 34. The embodiment shown in FIG. 10 controls a first set of opposed blades 26a and 26b and a second set of opposed blades 28a and 28b. In an alternate embodiment, collimator 22 has a generally circular aperture, with a corresponding blade structure for sizing. The rectangular aperture 34 provided using opposed sets of blades 26a, 26b, 28a, and 28b is advantaged because it not only allows aperture sizing, but also allows some measure of shift for the center of the beam that is provided. Collimator 22 blades are opaque to x-ray radiation, but this opacity can be over a range of values, from highly opaque to somewhat less opaque. Lower opacity allows some portion of the anatomy outside of the collimator aperture to be imaged, such as to provide a reference frame, for example. Using lower opacity collimator blades, for example, the background region 62 can be updated using lower radiation levels and provide an image of lower contrast, sharpness, or resolution than the ROI. This provides an alternative to completely blocking all image content except that within the ROI and allows the displayed image to relate the ROI to surrounding anatomy.

Other aspects of fluoroscopy system operation can also be controlled using GUI 72. The schematic diagram of FIG. 11 shows components for control of source 20 and DR receiver 50 positioning according to an embodiment of the present invention. Operator instructions on GUI 72 shift the relative position and, optionally, the size, of ROI 70, such as moving downward and to the right in the example shown. For slight positional adjustments, the rectangular collimator 22 of FIG. 10 can be used to shift the position of the imaging radiation toward ROI 70. For more pronounced positional shifts, however, it can be desirable to readjust the positions of source 20 and receiver 50. Collimator 22 adjustments may also be made at the same time. Host processor 52 commands to imaging controller 90 instruct actuator 94 to make the change in source 20 and receiver 50 position and orientation.

During sizing and position changes of the ROI 70 on GUI 72, it may be advantageous to display some portion of background region 62, as shown in the examples of FIGS. 10 and 11. Alternately, the display of ROI 70 can be resized to fill the display screen. Pan, zoom in, zoom out, and other functions are available to the operator for control of the displayed ROI 70 image.

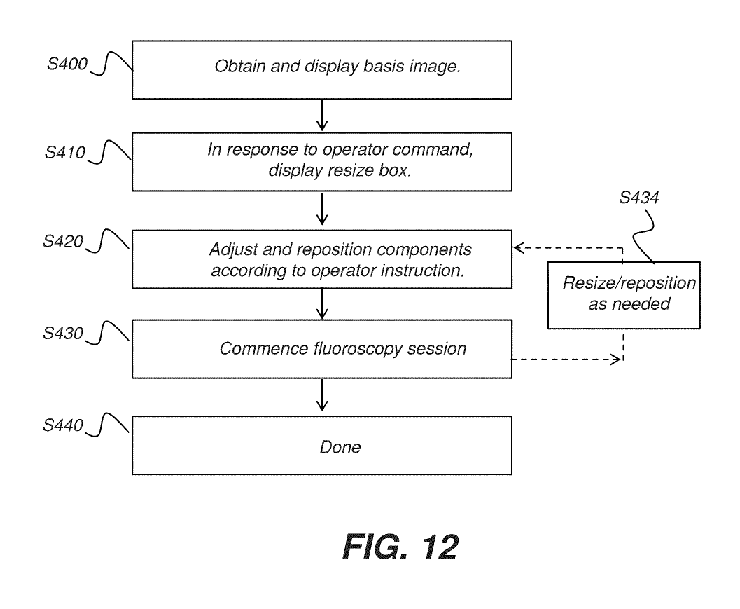

The logic flow diagram of FIG. 12 shows a sequence of steps for controlling devices of the fluoroscopy system according to operator instructions entered using GUI 72 as was shown in the examples of FIGS. 10 and 11. In an obtain basis image step S400, the initial base image or “scout image” for the fluoroscopy sequence is acquired and displayed. A command-specific response step S410 sets up the GUI so that it is enabled to receive operator instructions, such as for ROI resizing as noted in the example of FIG. 12. Step S410 may be automatically executed or may require specific operator instructions that specify the type of ROI manipulation that is desired. An adjustment step S420 accepts the operator instruction, such as for ROI resizing, and provides instruction parameters to host processor 52 (FIGS. 10 and 11). Components, such as collimator 22, are adjusted or reconfigured accordingly. Imaging then takes place in a fluoroscopic examination step S430, in which a sequence of images of the ROI is obtained, as described previously.

The sequence of FIG. 12 allows readjustment of the imaging apparatus during the fluoroscopy exam, according to operator interaction. An optional readjustment step S434 is executed when the operator enters an instruction that indicates the need for resizing, repositioning, or other reconfiguration of the fluoroscopy system. The operator instructions are not entered on the initial basis image 64 (FIGS. 10 and 11), but are entered directly on the GUI with reference to the ROI that is currently being displayed. Operator instructions can be entered using touch screen or other pointer utilities, including the gaze-tracking mechanism described previously with reference to FIG. 8B. According to an embodiment of the present invention, gaze-tracking mode is entered automatically upon commencement of the fluoroscopy session. According to an alternate embodiment, an operator instruction, such as entered on one of control buttons 74a-74d as shown previously in FIGS. 6A-6D, for example, activates a gaze-tracking mechanism for accepting operator instructions based on changes of operator focus. A completion step S440 indicates the end of the fluoroscopy exam.

In general, the image data content for fluoroscopic viewing is optimized for presentation, rather than for processing. This type of treatment can relate to how images are stored and processed in DICOM (Digital Imaging and Communications in Medicine) imaging apparatus.

In one exemplary embodiment, there can be one or more discontinuous background regions and/or regions of interest.

In addition, while a particular feature of an embodiment has been disclosed with respect to only one of several implementations or embodiments, such feature can be combined with one or more other features of the other implementations and/or other exemplary embodiments as can be desired and advantageous for any given or particular function. To the extent that the terms “including,” “includes,” “having,” “has,” “with,” or variants thereof are used in either the detailed description and the claims, such terms are intended to be inclusive in a manner similar to the term “comprising.” The term “at least one of” is used to mean one or more of the listed items can be selected. Further, in the discussion and claims herein, the term “exemplary” indicates the description is used as an example, rather than implying that it is an ideal.

The invention has been described in detail with particular reference to a presently preferred embodiment, but it will be understood that variations and modifications can be effected within the spirit and scope of the invention. The presently disclosed embodiments are therefore considered in all respects to be illustrative and not restrictive. The scope of the invention is indicated by the appended claims, and all changes that come within the meaning and range of equivalents thereof are intended to be embraced therein.

What is claimed is:

1. A method for acquiring a fluoroscopic image of a subject, comprising:
displaying a basis image from a fluoroscopic imaging system;
accessing one or more viewer instructions;
defining a region of interest for the displayed basis image responsive to the one or more viewer instructions; and
adjusting the position of one or more components of the fluoroscopic imaging system responsive to the one or more viewer instructions.

2. The method of claim 1 further comprising displaying the defined region of interest within the basis image and obtaining image data from one or more exposures, wherein the region of interest from the obtained image data displays at higher contrast than background portions of the basis image outside the region of interest.

3. The method of claim 2 further comprising updating the displayed background portions of the basis image according to exposure of the subject.

4. The method of claim 1 wherein the one or more viewer instructions are indicated on the displayed basis image using a touch screen.

5. The method of claim 1 wherein the one or more adjusted components control the size or position of a collimator opening.

6. The method of claim 5 wherein the one or more adjusted components further control the position of a radiation source or an imaging detector.

7. The method of claim 1 wherein the one or more viewer instructions are indicated on the displayed basis image using a mouse or other pointer.

8. The method of claim 1 wherein the one or more viewer instructions are indicated relative to the displayed basis image, and the one or more viewer instructions define a rectangular region of interest.

9. The method of claim 1 wherein the one or more viewer instructions are indicated relative to the displayed basis image, and the one or more viewer instructions define a non-rectangular region of interest.

10. The method of claim 1 further comprising shifting the position of the region of interest according to operator gaze.

11. A method for acquiring a sequence of fluoroscopic images of a subject, comprising:
displaying a basis image from a fluoroscopic imaging system;
defining a region of interest within the displayed basis image responsive to one or more viewer instructions entered on the displayed basis image using a touch screen;
adjusting a collimator of the fluoroscopic imaging system according to the one or more viewer instructions;
displaying the defined region of interest within the basis image; and
obtaining image data from one or more exposures, wherein the region of interest from the obtained image data displays at one or both of higher contrast and higher resolution than background portions of the basis image outside the region of interest.

12. The method of claim 11 further comprising tracking the attention of a viewer and adjusting the position of the region of interest according to changes in user attention.

13. The method of claim 11 further comprising updating one or more background portions of the basis image.

14. The method of claim 11 wherein adjusting the collimator comprises moving one or more collimator blades.

15. The method of claim 11 further comprising adjusting the position of the region of interest according to a viewer instruction or gesture.

16. A method for acquiring a sequence of fluoroscopic images of a subject, comprising:
displaying a basis image from a fluoroscopic imaging system;
defining a region of interest within the displayed basis image responsive to one or more viewer instructions entered on the displayed basis image using a touch screen;
adjusting a collimator of the fluoroscopic imaging system according to the one or more viewer instructions;
displaying the defined region of interest within the basis image; and
obtaining image data from one or more exposures, wherein the image data includes data for updating both the region of interest and one or more background portions of the basis image disposed outside the region of interest,
wherein the region of interest from the obtained image data displays at one or both of higher contrast and higher resolution than the background portions of the basis image disposed outside the region of interest.

17. The method of claim 16 wherein adjusting the collimator comprises moving one or more collimator blades.

18. The method of claim 16 further comprising adjusting the position of the region of interest according to a viewer instruction.

19. The method of claim 16 further comprising adjusting the position of the region of interest according to a viewer gesture.
